(19) United States
(12) Patent Application Publication
(10) Pub. No.: US 2024/0343693 A1
(43) Pub. Date: Oct. 17, 2024

(54) PREPARATION OF UREIDO METHACRYLATE

(71) Applicant: Rohm and Haas Company, Collegeville, PA (US)

(72) Inventor: Muhunthan Sathiosatham, Chalfont, PA (US)

(21) Appl. No.: 18/683,444

(22) PCT Filed: Aug. 16, 2022
(86) PCT No.: PCT/US2022/040389

§ 371 Date: Feb. 13, 2024

Related U.S. Application Data
(63) Continuation of application No. 63/233,448, filed on Aug. 16, 2021.

(51) Int. Cl. C07D 233/36

(52) U.S. Cl. CPC C07D 233/36

(57) ABSTRACT

The present invention relates to a method for preparing ureido methacrylate comprising the step of contacting, in the presence zinc acetylacetonate (Zn(acac)₂) and a radical inhibitor, methyl methacrylate with 1-(2-hydroxyethyl)imidazoline-2-one to form ureido methacrylate. The method provides an efficient and environmentally friendly way of making a key monomer used in the synthesis of acrylic latexes.

### BACKGROUND OF THE INVENTION

The present invention relates to the preparation of ureido methacrylates and analogs thereof. Ureido methacrylate is useful as a monomer in the synthesis of acrylic latexes. Latexes functionalized with ureido methacrylate groups have been found to be useful as adhesion promoters. (See U.S. Pat. No. 9,212,292 B2.)

Ureido methacrylate is commercially prepared by transesterification of 1-(2-hydroxyethyl)imidazoline-2-one and methyl methacrylate in the presence of a tin catalyst, most notably dibutyltin oxide. Although the reaction is quite efficient, concerns over the toxicity of tin, especially where the ensuing latex is used in food contact applications, create a motivation to find a safer and environmentally friendly synthesis approach, while maintaining process efficiency.

### SUMMARY OF THE INVENTION

The present invention addresses a need in the art by providing a method for preparing ureido methacrylate comprising the step of contacting, in the presence of zinc acetylacetonate and a radical inhibitor, methyl methacrylate with 1-(2-hydroxyethyl) imidazoline-2-one to form ureido methacrylate.

The method of the present invention provides a highly efficient way of producing ureido methacrylate in the presence of a catalyst that is more suitable to meet the demands of environmental, health, and safety standards.

### DETAILED DESCRIPTION OF THE INVENTION

The present invention is a method for preparing ureido methacrylate comprising the step of contacting, in the presence zinc acetylacetonate (Zn(acac)₂) and a radical inhibitor, methyl methacrylate with 1-(2-hydroxyethyl)imidazoline-2-one to form ureido methacrylate. Methyl methacrylate, 1-(2-hydroxyethyl)imidazoline-2-one, and Zn(acac)₂ are advantageously contacted together in the presence of a suitable solvent at a temperature in the range of from 50° C., or from 80° C., or from 100° C., to 150° C. or to 120° C. for sufficient time to achieve a conversion of 1-(2-hydroxyethyl)imidazoline-2-one of at least 80% or at least 90%.

Preferably, methyl methacrylate is used in a stoichiometric excess so that it serves as both a starting material and a solvent. Upon completion or near completion of the reaction, the solution of ureido methacrylate in methyl methacrylate may be used as is as a co-monomer; alternatively, it may be desirable to distil off at least some of the unreacted methyl methacrylate to leave a more concentrated solution of ureido methacrylate in methyl methacrylate. (Ureido methacrylate is commonly supplied commercially diluted in water as well as in methyl methacrylate.)

The mole:mole ratio of 1-(2-hydroxyethyl)imidazoline-2-one to Zn(acac)₂ is typically in the range of from 100:0.2, or from 100:0.5, or from 100:0.8, to 100:10, or to 100:5, or to 100:2. The mole:mole ratio of methyl methacrylate to 1-(2-hydroxyethyl)imidazoline-2-one is typically in the range of from 1.5:1, or from 2:1, to 10:1 or to 7:1 or to 5:1. Solvents other than methyl methacrylate may be used including reactive and non-reactive solvents. Examples of such solvents include those listed in U.S. Pat. No. 8,865,931 B2, col. 6, lines 55-68.

The reaction is advantageously carried out in the presence of a radical inhibitor at a concentration in the range of 50 ppm to 1000 ppm, based on the concentration of methyl methacrylate. Examples of suitable radical inhibitors include hydroquinone, hydroquinone monomethyl ether, phenothiazine, diethylhydroxylamine, 2-t-butyl-4-methylphenol, 6-t-butyl-2,4-dimethylphenol, di-t-butylcatechol, 4-hydroxy 2,2,6,6-tetramethyl-piperidin N-oxyl (4-hydroxy TEMPO), and 4-oxo-2,2,6,6-tetramethyl-piperidin N-oxyl.

It has been discovered that high yields of ureido methacrylate can be achieved in the presence of Zn(acac)₂ without any ancillary metal salts, including halo-metal salts such as LiCl, LiI, ZnCl₂, and metal carbonate salts such as Cs₂CO₃. Accordingly, in another aspect of the present invention, methyl methacrylate, 1-(2-hydroxyethyl)imidazoline-2-one, and Zn(acac)₂ are contacted together under reaction conditions in the absence or substantial absence of halo-metal or carbonate-metal salts, more particularly in the substantial absence of LiCl, LiI, ZnCl₂, or Cs₂CO₃. As used herein, “substantial absence” of the ancillary salt means that the mole:mole ratio of Zn(acac)₂ to the ancillary salt is at least 5:1 or at least 10:1, or at least 100:1, or at least 1000:1.

### EXAMPLE

### Preparation of Ureido Methacrylate

A 4-neck round bottom flask equipped with a stirrer, thermocouple, 1″, 10-tray Oldershaw column, and a heating mantle was charged with methyl methacrylate (601.5 g, 6 mol), 1-(2-hydroxyethyl)imidazoline-2-one (260.3 g, 2 mol), Zn(acac)₂ monohydrate (5.3 g, 0.02 mol), 4-hydroxy TEMPO (0.12 g) and hydroquinone monomethyl ether (0.52 g). The reaction mixture was heated to 110° C. at atmospheric pressure, and the distillate was collected when the vapor temperature at the top of the column was at 67° C., and the reflux splitter was at 3:7 (3 forward, 7 reflux). Over 3.5 h, approximately 94 g of the distillate was collected. ¹H-NMR spectroscopic analyses of the reaction mixture showed >93% conversion of 1-(2-hydroxyethyl)imidazoline-2-one, Michael adducts were less than 1%. Conversion was purposefully targeted at 93% to 95%; at higher conversion, it has been discovered that, at higher conversion, the desired product can react with methyl methacrylate to form an impurity that causes gelation in a subsequent emulsion polymerization using ureido methacrylate as a precursor.

The Oldershaw column was replaced with a straight distillation head, whereupon the pressure was reduced to 100 mm Hg and the pot contents heated to 53° C. The vacuum was slowly reduced to 2 mm Hg, and the pot temperature was slowly increased to 90° C. Approximately 360 g of excess MMA was distilled off. 1-(2-hydroxyethyl)imidazoline-2-one conversion determined to be 93.5%, Michael adducts, 0.8%, and residual methyl methacrylate, 3.4% as determined by ¹H-NMR spectroscopy.

1. A method for preparing ureido methacrylate comprising the step of contacting, in the presence zinc acetylacetonate (Zn(acac)₂) and a radical inhibitor, methyl methacrylate with 1-(2-hydroxyethyl)imidazoline-2-one to form ureido methacrylate.

2. The method of claim 1 wherein the mole: mole ratio of methyl methacrylate to 1-(2-hydroxyethyl)imidazoline-2-one is in the range of from 1.5:1 to 10:1; and the reaction is carried out at a temperature in the range of from 50° C. to 150° C.

3. The method of claim 2 wherein the mole: mole ratio of methyl methacrylate to 1-(2-hydroxyethyl)imidazoline-2-one is in the range of from 2:1 to 5:1; and the mole:mole ratio of 1-(2-hydroxyethyl)imidazoline-2-one to Zn(acac)₂ is in the range of from 100:0.2 to 100:10.

4. The method of claim 3 wherein the mole: mole ratio of 1-(2-hydroxyethyl)imidazoline-2-one to Zn(acac)₂ is in the range of from 100:0.5 to 100:5.

5. The method of claim 2 wherein the reaction is carried out in the absence or substantial absence of an ancillary metal salt.

6. The method of claim 5 wherein the mole:mole ratio of Zn(acac)₂ to the ancillary metal salt is at least 10:1.

7. The method of claim 5 wherein the mole:mole ratio of Zn(acac)₂ to the ancillary metal salt is at least 100:1; and the ancillary metal salt is LiCl, LiI, ZnCl₂, and/or Cs₂CO₃.

8. The method of claim 5 wherein the reaction is carried out in the absence of any of the ancillary metal salts LiCl, LiI, ZnCl₂, and Cs₂CO₃.
